(19) United States
(12) Patent Application Publication
(10) Pub. No.: US 2021/0130253 A1
(43) Pub. Date: May 6, 2021

(54) PURIFICATION METHOD

(71) Applicant: Bayer AS, Oslo (NO)

(72) Inventors: Janne Olsen FRENVIK, Oslo (NO); Olav B. RYAN, Rasta (NO)

(73) Assignee: Bayer AS, Oslo (NO)

(21) Appl. No.: 16/903,181

(22) Filed: Jun. 16, 2020

Related U.S. Application Data
(63) Continuation of application No. 16/067,404, filed on Jun. 29, 2018, now abandoned.

(30) Foreign Application Priority Data
Jan. 5, 2016 (GB) 1600158.8

(51) Int. Cl. C07B 59/00; B01D 15/36; A61K 51/10; B01J 39/05; C07B 63/00; B01D 15/42; C07F 9/00

(52) U.S. Cl. CPC C07B 59/008; B01D 15/362; A61K 51/1051; B01J 39/05; C07B 2200/05; C07B 59/00; B01D 15/424; C07B 59/001; C07F 9/00; C07B 63/00

(57) ABSTRACT

The invention provides a method for the purification of complexed ²²⁷Th from a mixture comprising complexed ²²⁷Th and ²²³Ra (complexed or in solution), said method comprising:
i) preparing a first solution comprising a mixture of complexed ²²⁷Th ions and ²²³Ra ions in a first aqueous buffer;
ii) loading said first solution onto a separation material;
iii) eluting complexed ²²⁷Th from said separation material whereby to generate a second solution comprising complexed ²²⁷Th;
iv) Optionally rinsing said separation material using a first aqueous washing medium;

The invention additionally provides a purified ²²⁷Th solution, a pharmaceutical product and its use in treatment of disease such as cancer and a kit for generation of such a product.

### FIELD OF THE INVENTION

The present invention relates to the purification of components for radio-pharmaceuticals. In particular, the present invention relates to methods for the purification of complexed thorium-227 for endo-radionuclide therapy, particularly where that purification is carried out shortly prior to use in pharmaceutical administration to human subjects.

### BACKGROUND TO THE INVENTION

Specific cell killing can be essential for the successful treatment of a variety of diseases in mammalian subjects. Typical examples of this are in the treatment of malignant diseases such as sarcomas and carcinomas. However the selective elimination of certain ceil types can also play a key role in the treatment of many other diseases, especially immunological, hyperplastic and/or other neoplastic diseases.

The most common methods of selective treatment are currently surgery, chemotherapy and external beam irradiation. Targeted endo-radionuclide therapy is, however, a promising and developing area with the potential to deliver highly cytotoxic radiation to unwanted cell types.

The most common forms of radiopharmaceutical currently authorised for use in humans employ beta-emitting and/or gamma-emitting radionuclides. There has, however, been a recent surge in interest in the u se of alpha-emitting radionuclides in therapy because of their potential for more specific cell killing. One alpha-emitting nuclide in particular, radium-223 (²²³Ra) has proven remarkably effective, particularly for the treatment of diseases associated with the bone and bone-surface. Additional alpha-emitters are also being actively investigated and one isotope of particular interest is the alpha-emitter thorium-227.

The radiation range of typical alpha emitters in physiological surroundings is generally less than 100 micrometers, the equivalent of only a few cell diameters. This makes these nuclei well suited for the treatment of tumours, including micrometastases, because little of the radiated energy will pass beyond the target cells and thus damage to surrounding healthy tissue might be minimised (see Feinendegen et ah, Radiat Res 148:195-201 (1997)). In contrast, a beta particle has a range of 1 mm or more in water (see Wilbur, Antibody Immunocon Radiopharm 4: 85-96 (1991)).

The energy of alpha-particle radiation is high compared to beta particles, gamma rays and X-rays, typically being 5-8 MeV, or 5 to 10 times that of a beta particle and 20 or more times the energy of a gamma ray. Thus, this deposition of a large amount of energy over a very short distance gives α-radiation an exceptionally high linear energy transfer (LET), high relative biological efficacy (RBE) and low oxygen enhancement ratio (OER) compared to gamma and beta radiation (see Hall, “Radiobiology for the radiologist”, Fifth edition, Lippincott Williams & Wilkins, Philadelphia Pa., USA, 2000). These properties explain the exceptional cytotoxicity of alpha emitting radionuclides and also impose stringent demands on the level of purity required where an isotope is to be administered internally. This is especially the case where any contaminants may also be alpha-emitters, since these can potentially be retained in the body and cause significant damage. Radiochemical purity should be as high as reasonably feasible and contamination with non-targeted radionuclides should be minimised, particularly where the contaminant is an alpha-emitter.

The radioactive decay chain from ²²⁷Ac, generates ²²⁷Th and then leads to ²²³Ra and further radioactive isotopes. The first three isotopes in this chain are shown below. The table shows the element, molecular weight (Mw), decay mode (mode) and Half-life (in years (y) or days (d)) for ²²⁷Th and the isotopes preceding and following it. Preparation of ²²⁷Th can begin from ²²⁷Ac, which is itself found only in traces in uranium ores, being part of the natural decay chain originating at ²³⁵U. One ton of uranium ore contains about a tenth of a gram, of actinium and thus although ²²⁷Ac is found naturally, it is more commonly made by the neutron irradiation of ²²⁶Ra in a nuclear reactor.

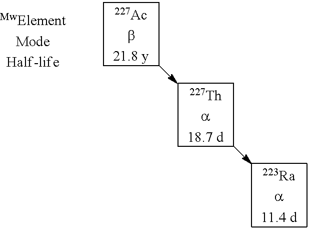

It can be seen from this illustration that ²²⁷Ac, with a half-life of over 20 years, is a very dangerous potential contaminant with regard to preparing ²²⁷Th from the above decay chain for pharmaceutical use. Even once the ²²⁷Ac is removed or reduced to a safe level, however, ²²⁷Th will continue to decay to ²²³Ra with a half-life of just under 19 days. Since ²²³Ra is an alkaline earth metal it will not easily be coordinated by ligands designed for thorium or other actinides. This ²²³Ra then forms the beginning of a potentially uncontrolled (untargeted) decay chain including 4 alpha-decays and 2 beta-decays before reaching stable ²⁰⁷Pb. These are illustrated in the table below:

| Nuclide | ²²⁷Th | ²²³Ra | ²¹⁹Rn | ²¹⁵Po | ²¹¹Pb | ²¹¹Bi | ²⁰⁷Tl | ²⁰⁷Pb |
|---|---|---|---|---|---|---|---|---|
| ½-life | 18.7 d | 11.4 d | 4.0 s | 1.8 ms | 36.1 m | 2.2 m | 4.8 m | stable |
| α-energy/ | 6.15 | 5.64 | 6.75 | 7.39 | | 6.55 | | |
| MeV | | | | | | | | |
| β-energy | | | | | 1.37 | | 1.42 | |
| (max)/MeV | | | | | | | | |
| Energy % | 17.5 | 16.0 | 19.1 | 21.0 | 3.9 | 18.6 | 4.0 | |

It is evident from the above two decay tables that ²²³Ra cannot be entirely eliminated from any preparation of ²²⁷Th because the latter will constantly be decaying and generating the former. It is clear, however, that more than 25 MeV in radiated energy will be released from the decay of each ²²³Ra nucleus administered to a patient, before that nucleus reaches a stable isotope. It is also probable that such ²²³Ra will not be bound and targeted by the systems of chelation and specific binding designed to transport ²²⁷Th to its site of action, due to the differing chemical nature of the two elements. Therefore for the purpose of targeted cell killing, maximising the therapeutic effect and minimising side-effects, it is important to have control over the level of ²²³Ra in any ²²⁷Th preparation prior to administration. Even where the product must be stored or transported for a period (e.g. 12 to 96 hours, such as up to 48 hours) before administration, it remains important to begin with very pure isotope in order still to minimise the contamination with daughter isotopes.

Separation of ²²⁷Th from ²²³Ra could be carried out quickly and conveniently in a radiological laboratory, such as at the site of generation by decay of ²²⁷Ac. However, this would not always be possible and may not achieve the desired result effectively because the resulting purified ²²⁷Th must then be transported to the site of administration. If this site of use is remote from the site of origin of the ²²⁷Th then a further build-up of ²²³Ra will occur during storage and transport.

Furthermore, the ²²⁷Th can be purified from ²²³Ra either before or after complexation of the ²²⁷Th to form a pharmaceutical (such as a targeted radiopharmaceutical complex). Purification may be simpler if the method is earned out prior to the complexation, particularly if the ligand is conjugated to a targeting molecule such as an antibody because the large conjugate may be difficult to handle. However, if a method of purification can be devised that can be used at or close to the point-of-care and which can used to separate undesirable ²²³Ra from a pharmaceutical ²²⁷Th complex (e.g. a targeted complex) then this would provide a considerable advantage because no delay between purification and administration is required for generation for the complex.

In view of the above, it would be a considerable advantage to provide a method of purifying ²²⁷Th from contaminant ²²³Ra which could be carried out at a centralized location from which the purified ²²⁷Th can reach the site of administration significantly more quickly than the half-life of the isotope. Where the purified isotope will be stored from some time (e.g. 12 to 96 hours) then the method should provide a very high degree of removal of ²²³Ra so that only radium caused by unavoidable in-growth is administered to the subject. Alternatively, purification may take place at or close to the point-of-care, at or shortly before the time of administration utilising a simple method that would not require extensive training and experience to carry out. In either embodiment, the method should be robust, reliable and effective, since the resulting purified ²²⁷Th (²²⁷Th complex) may be used directly in pharmaceutical preparation.

It would be a further advantage if this method could be earned out on complexed ²²⁷Th, preferably conjugated to a targeting moiety, it would be an advantage if the use of strong mineral acids and/or strong bases could be avoided from a safety and handling point of view (as well as avoiding possible degradation of sensitive components such as targeting moieties of a targeting complex) and also since this avoids the need for separation of these materials from the final product. This applies particularly if the reagents used are suitable for direct use in the final drug product since speed is thereby maximised. It would also be an advantage if small volumes could be used to ease handling and reduce the volume of contaminated waste. It would be a further advantage if this method could be implemented with a simple group of reagents and items of apparatus, which could be supplied for such a contemporaneous preparation, optionally in the form of a kit.

Previously known preparations for ²²⁷Th have generally been for laboratory use and/or not tested for purity to pharmaceutical standards. In WO2004/091668, for example, ²²⁷Th was prepared by anion exchange from a single column and used for experimental purposes without validation of the purity. The primary aim of separation in most preparative methods for ²²⁷Th has been the removal of the long-lived ²²⁷Ac parent isotope. Methods have not previously been devised or optimised for removal of ²²³Ra which has grown-in in a ²²⁷Th sample previously purified from ²²⁷Ac.

### BRIEF DESCRIPTION OF THE INVENTION

The present inventors have now established that a quick and simple purification procedure may be used to remove ²²³Ra from a preparation of ²²⁷Th. This method allows for the ²²⁷Th to be in complexed form and even be complexed and conjugated to a targeting moiety such as an antibody. The method may use a single purification step. In this way, a ²²⁷Th solution of very high radiochemical purity may be produced while providing a number of desirable advantages in the method, particularly with regard to the reduced delay between purification and administration and less handling of the product at the site of administration.

In a first aspect, the present invention therefore provides a method for the purification of complexed ²²⁷Th from a mixture comprising complexed ²²⁷Th and ²²³Ra (complexed or in solution), said method comprising:
i) preparing a first solution comprising a mixture of complexed ²²⁷Th ions and ²²³Ra ions in a first aqueous buffer;
ii) loading said first solution onto a separation material such as a strong cation exchange resin;
iii) eluting complexed ²²⁷Th from said separation material whereby to generate a second solution comprising complexed ²²⁷Th;
iv) optionally rinsing said separation material using a first aqueous washing medium;

Generally the steps i) to iv) will be earned out in the order given above, although other steps and processes may evidently be carried out during or between the listed steps.

The process will optionally and preferably also include the following step prior to step i) above:
X) contacting ²²⁷Th ions with at least one complexing agent in solution, whereby to form at least one aqueous solution of complexed ²²⁷Th. Preferably the complexing agent will be a chelating moiety conjugated (e.g. covalently conjugated) to a targeting moiety, such as those described herein.

The process will optionally also include at least one of the following further steps, each generally conducted after steps i) to iv) above:
v) assaying for the ²²⁷Th content of said second solution;
vi) evaporating the liquid from said second solution;
vii) forming at least one radiopharmaceutical formulation at least a portion of the complexed ²²⁷Th contained in said second solution;
viii) sterile filtering said radiopharmaceutical.

Step vii) forms a particularly preferably additional step.

In a further aspect, the present invention provides a solution or other sample of ²²⁷Th comprising less than 50 KBq ²²³Ra per 1 MBq ²²⁷Th, preferably less than 10 KBq ²²³Ra per 1 MBq ²²⁷Th. Such a solution is optionally formed or formable by any of the methods herein described, and is preferably formed or formable by the preferred methods herein described. Correspondingly, the methods of the invention are preferably for the formation of a solution of ²²⁷Th comprising less than 50 KBq ²²³Ra per 1 MBq ²²⁷Th, preferably less than 10 KBq ²²³Ra per 1 MBq ²²⁷Th. A corresponding pharmaceutical preparation is also provided, which may be sterile and may comprise at least one complexing agent (especially for ²²⁷Th), at least one targeting agent (e.g. conjugated to said complexing agent), and optionally at least one pharmaceutically acceptable carrier or diluent.

In a still further aspect, the invention also provides a kit (typically a kit for carrying out a method of the invention) comprising a mixture of ²²⁷Th and ²²³Ra, a first aqueous buffer, a chelator (preferably conjugated or congatable to a targeting moiety) and a separation material (e.g. cation exchange resin). The mixture of ²²⁷Th and ²²³Ra (as with the first solution in other aspects of the invention) will typically also comprise further ²²³Ra daughter products. Such a mixture may be the result of radioactive decay of purified or partially purified ²²⁷Th (optionally complexed or in solution) during storage and transportation.

### DETAILED DESCRIPTION OF THE INVENTION

Pharmaceuticals of all types must routinely be produced to a very high, standard of purity and a very high confidence that standards (e.g. of purity and sterility) have been met. Administration of an alpha-emitting radionuclide to the body of a subject requires all of these considerations but additionally adds a need for high radiochemical purity. Purification from long-lived precursor isotopes is one key aspect of radiochemical purity but this can typically be accomplished in a specialist radiochemical laboratory or factory where complex methods and handling procedures can be utilised.

A further level of radiochemical purification may be necessary, however, in the event that the radionuclide of interest decays to other radioactive isotopes. The generation of radioactive daughter isotopes may contribute significantly to the toxicity of endo-radionuclide therapy and can be dose-limiting. In the case of ²²⁷Th, the daughter isotope is radium, an alkaline earth metal, while the parent is a transition metal of the actinide series. This means that any chelation or complexation which may have been suitable for binding thorium, will probably not be chemically suitable for retaining the daughter radium. Alpha decay additionally imparts a very significant “recoil” energy onto the daughter nucleus as a result of conservation of momentum following ejection, of an alpha particle at very high speeds. This recoil carries many times more energy than a covalent bond or coordinating interaction and will inevitably shunt the daughter nucleus out of the immediate environment of the original parent isotope.

Since the presence of ²²³Ra and its daughters generated in vivo by ²²⁷Th decay is potentially dose-limiting, it is important that no additional, unnecessary, ²²³Ra is administered to the subject to further limit the acceptable therapeutic dose of ²²⁷Th or to exaggerate the side effects.

The present invention has been developed in view of the inevitable in-growth of ²²³Ra into a ²²⁷Th sample and the desire to minimise that ²²³Ra delivered to the subject, as far as reasonably possible. Since ²²³Ra will initially grow in at a rate of around 0.2% of the total activity per hour, the method should be carried out no more than a few hours (e.g. within 72 hours or within 48 hours) before administration in order to minimise the unnecessary dose. Similarly, if the ²²⁷Th can be used within 2-4 hours of preparation then the method should preferably provide ²²⁷Th with around 99% (e.g. 95% to 99.9%) radiochemical purity with respect to ²²³Ra (at the time of preparation). Higher purity may be inefficient and/or insignificant since ingrowth before use will undo any benefits of a more stringent purification method while lower purity (say less than 90% or less than 95% radiochemical purity) is undesirable because the dose of ²²³Ra (and thus toxicity) could reasonably be further limited while allowing for a realistic administration time.

In one embodiment, the mixtures of ²²⁷Th and ²²³Ra for use in the present invention will contain no significant amount of radioactive isotopes that are not in the decay chain beginning at ²²⁷Th. In particular, the mixtures of ²²⁷Th. and ²²³Ra for use in any of the aspects of the present invention will preferably comprise less than 20 Bq ²²⁷Ac per 100 MBq ²²⁷Th, preferably less than 5 Bq ²²¹Ac per 100 MBq ²²⁷Th.

The present invention provides a method for the production of ²²⁷Th at a purity level suitable for use in endo-radionuclide therapy. The added benefit of the present method is that it may be carried out on ²²⁷Th that is already complexed to a chelator (preferably a chelator which is conjugated to a targeting moiety). By carrying out the separation on a pre-complexed sample, the method reduces the number of further step that are required for generation of a pharmaceutical formulation and thus allows administration of the isotope more quickly after purification. Since radioisotopes continue to decay under all storage conditions, purification shortly before administration allows for a pharmaceutical with greater isotopic purity. In the present invention complexed ²²⁷Th, typically in the form of an ion complexed to a ligand conjugated to a targeting moiety (known as a “targeted thorium conjugate”—TTC) is purified directly, preferably shortly before administration.

A number of preferred features of the system are indicated below, each of which may be used in combination with any other feature where technically viable, unless explicitly indicated otherwise. The methods and all corresponding embodiments of the invention will preferably be carried out on a scale suitable for patient administration. This scale may be that if a single therapeutic dose, or may be that suitable for a number of subjects, each receiving a dose. Typically the method will be used at a scale suitable for administration within 1 to 5 hours, such as around 1 to 10 typical doses of ²²⁷Th. Single-dose purification forms one preferred embodiment. Evidently, a typical dose will depend upon the application, but it is anticipated that a typical dose may be from 0.5 to 200 MBq, preferably 1 to 25 MBq, and most preferably around 1.2 to 10 MBq. Pooled dosage purification will be carried out where possible, using up to 20, preferably up to 10 or up to 5 typical doses. Purification my thus be carried out with up to 200 MBq, preferably up to 100 MBq and divided into separate doses after purification, as appropriate.

Step i) of the method of the invention relates to solution comprising ²²⁷Th and ²²³Ra (and will commonly also comprise ²²³Ra daughter isotopes—see those tabulated above). Such a mixture will inherently form, by the gradual decay of a sample of ²²⁷Th, but for use in the invention will preferably also have one or more of the following features, either individually or in any viable combination:
a) The ²²⁷Th radioactivity may be at least 0.5 MBq (e.g. 0.5 MBq to 100 MBq), preferably at least 0.5 MBq, more preferably at least 1.4 MBq;
b) The solution may be formed in a first aqueous buffer solution;
c) The solution may have a volume of no more than 50 ml (e.g. 0.1 to 20 ml or 0.1 to 10 ml), preferably no more than 10 ml or 5 ml, more preferably between 3 and 7 ml.
d) The first aqueous buffer solution may be at a pH of between 3 and 6.5, preferably between 3.5 and 6, and particularly between 4 and 6.
e) The first aqueous buffer solution may be used at a concentration of 0.01 to 0.5M, such as 0.03 to 0.05M or 0.1 to 0.2 M.
f) The first aqueous buffer solution may comprise, consist essentially of or consist of at least one organic acid buffer.
g) The first aqueous buffer solution may comprise, consist essentially of or consist of at least one organic acid buffer selected from citrate buffer, acetate buffer and mixtures thereof.
h) The first aqueous buffer solution may optionally additionally comprise at least one free radical scavenger and/or at least one chelating agent (especially a non-buffering chelating agent). Many of each are known in the art and include pABA (scavenger) and EDTA (chelator).
i) The first aqueous buffer solution may optionally additionally comprise other additives including salts, such as NaCl.

Step ii) of the method of the invention relates to the loading of the first solution onto a separation material (such as a cation exchange resin). This step and the entities referred to therein may have the following preferable features, either individually or in any viable combination, and optionally in any viable combination with any of the features of the other steps as described herein:
a) The separation material may be a cation exchange resin or hydroxyapatite, preferably a strong cation exchange resin.
b) The resin (e.g. cation exchange resin) may be silica based resin;
c) The cation exchange resin may comprise one or more acid functional groups;
d) The cation exchange resin may comprise at least one acid moiety and preferably at least one carboxylic acid or sulphonic acid moiety, such as an alkyl sulphonic acid resin such as a propylsulphonic acid (PSA) resin;
e) The resin (e.g. strong cation exchange resin) may have an average particle size of 5 to 500 μm, preferably 10 to 200 μm.
f) The separation material (e.g. cation exchange resin) may be used in the form of a column.
g) The amount of separation material (e.g. resin) used (e.g. when packed in a column) may be 100 mg or less, (e.g. 2 to 50 mg), preferably 10 to 50 mg.
h) The separation material (e.g. resin) may be pre-conditioned by washing with one or more volumes of an aqueous medium prior to loading with the first solution. Generally a buffer solution, and more preferably the first aqueous buffer will be used for pre-conditioning.

Step iii) of the method of the invention relates to eluting complexed ²²⁷Th from the separation material (e.g. strong cation exchange resin) whereby to generate a second solution comprising complexed ²²⁷Th. This step and the entities referred to therein may have the following preferable features, either individually or in any viable combination, and optionally in any viable combination with any of the features of the other steps as described herein:
a) The elution may be by means of an eluent solution or by means of “dry” elution, such as by elution under gravity, under centrifugal force or under gas pressure from above and/or vacuum from below.
b) Where elution is by means of an eluent solution, this may be an aqueous buffer solution, such as any of those described herein, including organic acid buffer solutions;
c) The elution may be by “dry” means, preferably under gravitational or centrifugal force, such as spinning in a centrifuge.
d) elution by centrifugal force may be at a “relative centrifugal force” (RCF) of at least 1000, preferably at least 2000 or at least 5000 times the force of gravity (e.g. an ref of 1000 to 50000 g) for a period of 10 seconds to 10 minutes, preferably 20 seconds to 5 minutes;

Step iv) of the method of the invention relates to the optional step of rinsing said separation material (e.g. strong cation exchange resin) using a first aqueous washing medium. This step and the entities referred to therein may have the following preferable features, either individually or in any viable combination, and optionally in any viable combination with any of the features of the other steps as described herein:
a) The first aqueous washing medium may be water, such as distilled water, deionised water or water for injections or may be a buffer such as an organic acid buffer as described herein;
b) The first aqueous washing medium may comprise the same buffer as the first buffer solution;
c) The optional washing step may be omitted;
d) The optional washing step may comprise adding a first washing medium to the resin following “dry” elution as described here and then “dry” eluting the washing medium, such as by gravity or centrifugation; or under gas pressure from above and/or vacuum from below.
e) The solution eluted in the washing step may be combined with the second solution comprising ²²⁷Th.

Following step iv) of the method, of the invention, the separation material (e.g. resin) will typically be disposed of as radioactive waste. Since the amount of resin required is typically quite small (e.g. less than 50 mg), this does not present a major disposal issue. If, however, it is desired to re-use the resin or to recover the ²²³Ra for assay or any other reason, the ²²³Ra may be eluted using any suitable medium. Suitable media for such recovery include buffer solutions, such as those described herein and aqueous mineral acids, such as HCl and H₂SO₄. If the resin is to be re-used then it will typically be regenerated with several volumes of the first buffer solution prior to re-use.

The methods of the present invention may comprise a number of optional steps, each of which may be present or absent independently so far as technically possible.

Prior to the separation steps i) to iv), it is preferred to include optional preparation step X). Step X) comprises the preparation of complexed ²²⁷Th from ²²⁷Th ion and a chelator. Preferably the complexing agent will comprise a chelator conjugated (e.g. by covalent bonding) to a targeting moiety. This step and the entities referred to therein may have the following preferable features, either individually or in any viable combination, and optionally in any viable combination with any of the features of the other steps as described herein. Furthermore, all of the features of the radiopharmaceutical indicated herein form preferred features of the pharmaceutical aspect of the present invention, particularly where that pharmaceutical is formed or formable by a method of the invention:
a) The amount of ²²⁷Th contacted with the chelator may be 1 MBq to 100 MBq, preferably from 1 to 10 MBq.
b) The complexing agent may comprise an octadentate ligand.
c) The complexing agent may comprise a hydroxypyridinone such hydroxypyridinone (HOPO) ligand, preferably an octadentate 3,2-hydroxypyridinone (3,2-HOPO).
d) The complexing agent may comprise a targeting moiety, preferably conjugated to an octadentate ligand such as a HOPO ligand (e.g. a 3,2-HOPO).
e) The targeting moiety may be an antibody, antibody construct, antibody fragment (e.g. FAB or F(AB)′2 fragment or any fragment comprising at least one antigen binding region(s)), or a construct of such fragments.
f) The targeting moiety may be a, receptor or receptor binder (e.g. a hormone, vitamin, folate or a folate analogue) a bisphosphonate or nano-particle.
g) The targeting moiety may have specificity for at least on disease-associated antigen such as a “cluster of differentiation” (CD) cell surface molecule (e.g. CD22, CD33, CD34, CD44, CD45, CD166 etc).
h) The targeting moiety may be linked to the ligand by a covalent linker whereby to form complexing agent in the form of a targeting conjugate.
j) The contacting may comprise incubating the ²²⁷Th ions in solution with the complexing agent (especially targeting conjugate). Such incubation may be at a temperature below 50° C., preferably 10 to 40° C., such as 20 to 30° C. Such incubation may be for a period of less than 2 hours, such as 1 minute to 60 minutes (e.g. 1 to 15 minutes), preferably 15 to 45 minutes.
k) The contacting may be in a buffer solution, preferably in said first buffer solution.

Step v) of the method of the invention relates to optionally assaying for the ²²⁷Th content of the second solution. This step and the entities referred to therein may have the following preferable features, either individually or in any viable combination, and optionally in any viable combination with any of the features of the other steps as described herein:
a) ²²⁷Th may be assayed by gamma detection/spectroscopy, such as by use of a germanium semiconductor detector (high purity germanium detector—HPGe);
b) ²²⁷Th content may be compared to a desired pharmaceutical dose and diluted to a standard concentration, or an appropriate dose withdrawn for administration.

Step vi) of the method of the invention relates to the optional step of evaporating the liquid from said second solution. This step may be desirable where the final pharmaceutical composition has a low volume. Typically, the first aqueous buffer will be selected such that it is compatible with the labelling reaction (as described herein) and is physiologically tolerable (i.e. suitable for injection at the concentrations and amounts used). In this way, multiple manipulations and changes of solvent, such as those involving concentration step vi) will preferably be avoided. Where necessary, this step may be included and the entities referred to therein may have the following preferable features, either individually or in any viable combination, and optionally in any viable combination with any of the features of the other steps as described herein:
a) The evaporation may be conducted under reduced pressure (e.g. 1 to 500 mbar).
b) The evaporation may be conducted at elevated temperature (e.g. 50 to 200° C., preferably 80 to 110° C.);

Step vii) of the method of the invention relates to the optional step of forming at least one radiopharmaceutical from at least a portion of the ²²⁷Th purified by means of steps i) to iv). This step and the entities referred to therein may have the following preferable features, either individually or in any viable combination, and optionally in any viable combination with any of the features of the other steps as described herein. Furthermore, all of the features of the radiopharmaceutical indicated herein form preferred features of the pharmaceutical aspect of the present invention, particularly where that pharmaceutical is formed or formable by a method of the invention:
a) The portion of the complexed ²²⁷Th from said, second solution (purified by means of steps i) to iv)) may be 1 MBq to 100 MBq, preferably from 1 to 10 MBq.
b) Pharmaceutical carriers, diluents, buffers, salts, preservatives etc may be added whereby to form an injectable radiopharmaceutical.
c) The complexed ²²⁷Th from said second solution may be diluted to a standard activity based upon the activity measurements obtained in step v), optionally correcting for the period between preparation (or measurement) and administration.
d) The radiopharmaceutical may be prepared at or near the point of care and/or may be for use within a short period (e.g. within 96 hours from purification to injection, preferably within 48 hours or within 36 hours of purification).

The radiopharmaceutical formed or formable in the various aspects of the present invention may be used in the treatment of any suitable disease, such as a neoplastic or hyperplastic disease (e.g. a carcinoma, sarcoma, melanoma, lymphoma, or leukemia). The pharmaceutical formulation, both as such and for such a use, as well as the corresponding methods of treatment of a subject form further aspects of the invention. Such a subject will typically be in need thereof, such as a subject suffering from a neoplastic or hyperplastic disease (e.g. those described herein). The invention will further provide for a method of administration of a radiopharmaceutical to a subject (e.g. one in need thereof) comprising forming said radiopharmaceutical by steps i) to iv), vii) and optionally any of steps v), vi) and/or viii) and administering said radiopharmaceutical (e.g. by intravenous injection or directly to a specific tissue or site) to said subject.

Step viii) of the method of the invention is an optional step comprising sterilising the solution or pharmaceutical (especially that formed in step vii)). This step and the entities referred to therein may have the following preferable features, either individually or in any viable combination, and optionally in any viable combination with any of the features of the other steps as described herein:
a) The sterilisation may be by heating, by irradiation or by filtration.
b) The filtration may be through a suitable membrane, such as a 0.22 μm (or smaller) membrane.
c) The filtration may be by syringe through a suitable syringe filter.

In addition to the above steps, the methods of the invention and all corresponding aspects may comprise additional steps, for example to validate the purity of the ²²⁷Th for pharmaceutical purposes, to exchange counter-ions, concentrate or dilute the solution or to control factors such as pH and ionic strengths. Each of these steps thus forms an optional but preferable additional step in the various aspects of the present invention.

It is preferable that the methods of the present invention provide tor a high yield of the complexed ²²⁷Th product. This is not only because of the desire to avoid wastage or a valuable product but also because all lost radioactive material forms radioactive waste which must then be disposed of safely. Thus, in one embodiment, at least 50% (e.g. 50 to 90% or 50% to 98%) of the ²²⁷Th loaded in step ii) is eluted in step iv). This will preferably be at least 70%, more preferably at least 80% and most preferably at least 85% yield. In a related aspect, at least 50% of the ²²⁷Th eluted in step iv) is eluted in the form of a ²²⁷Th complex (the remainder being eluted as uncomplexed ions in solution). This will preferably be at least 70% preferably at least 80% and more preferably at least 90%. In one preferred embodiment substantially 100% (e.g. at least 95%) of the ²²⁷Th eluted in step iv) is eluted in the form of a ²²⁷Th complex.

In a corresponding aspect of the present invention, there is additionally provided pharmaceutical composition comprising the complexed ²²⁷Th (especially purified as described herein) and optionally at least one pharmaceutically acceptable diluent. Such a pharmaceutical composition may comprise ²²⁷Th of a purity indicated herein, optionally formed or formable by the methods of the present invention. Suitable carriers and diluents including water for injection, pH adjusters and buffers, salts (e.g. NaCl) and other suitable materials will be well known to those of skill in the art.

The pharmaceutical composition will comprise the complexed ²²⁷Th as described here, typically as the complex of an ion, such as the Th⁴⁺ ion. Such compositions comprise a complex of ²²⁷Th of the invention with at least one ligand, such as an octadentate 3,2-hydroxypyridinone (2,3-HOPO) ligand. Suitable ligands are disclosed in WO2011/098611, which is hereby incorporated by reference, particularly with reference to formulae I to IX disclosed therein, which represent typical suitable HOPO ligands. Such ligands may be used in themselves or conjugated to at least one targeting moiety, such as an antibody. Most commonly the ligand will be conjugated to a targeting moiety prior to steps i) to iv) as described herein. Antibodies, antibody constructs, fragments of antibodies (e.g. FAB or F(AB)′2 fragments or any fragment comprising at least one antigen binding region(s)), constructs of fragments (e.g. single chain antibodies) or a mixture thereof are particularly preferred targeting moieties. The pharmaceutical compositions of the invention may thus comprise Th⁴⁺ ion complexed to a conjugate of a 3,2-hydroxypyridinone (3,2-HOPO) ligand and at least one antibody, antibody fragment or antibody construct, purified as described herein, with optionally pharmaceutically acceptable earners and/or diluents. The embodiments described herein with respect to the pharmaceutical composition will also form, embodiments of the corresponding method where practicable and vice versa.

As used herein, the term “comprising” is given an open meaning such that additional components may optionally be present (thus disclosing both “open” and “closed” forms). In contrast the term “consisting of” is given a closed meaning only, such that (to an effective, measurable and/or absolute degree), only those substances indicated (including any optional substances as appropriate) wall be present. Correspondingly, a mixture or substance described as “consisting essentially of” will in essence consist of the stated components such that any additional components do not affect the essential behaviour to any significant extent. Such mixtures may, for example, contain less than 5% (e.g. 0 to 5%) of other components, preferably less than 1% and more preferably less than 0.25% of other components. Similarly, where a term is given as “substantially”, “around”, “about” or “approximately” a given value, this allows for the exact value given, and independently allows for a small variability, particularly where this does not affect the substance of the property described. Such variability may be, for example ±5% (e.g. ±0.001% to 5%), preferably ±1%, more preferably ±0.25%.

The invention will now be illustrated further by reference to the following non-limiting examples and the attached figures, in which:

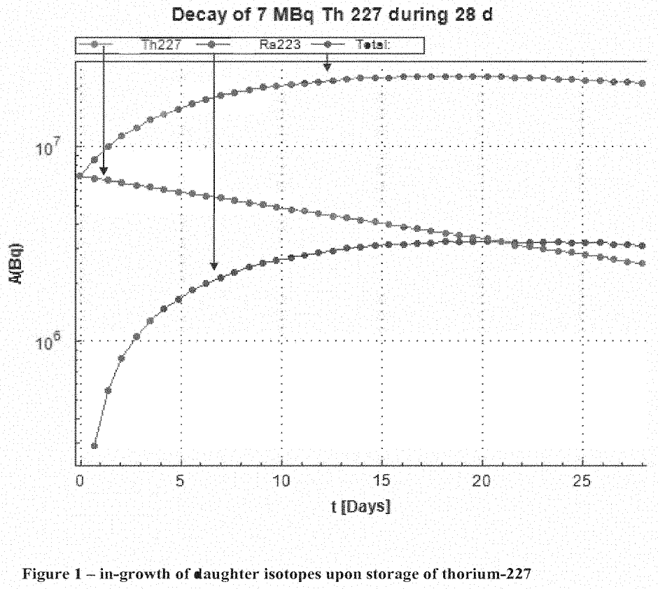

FIG. 1 Shows the decay of ²²⁷Th over time and the corresponding in-growth of ²²³Ra and daughter isotopes over 28 days.

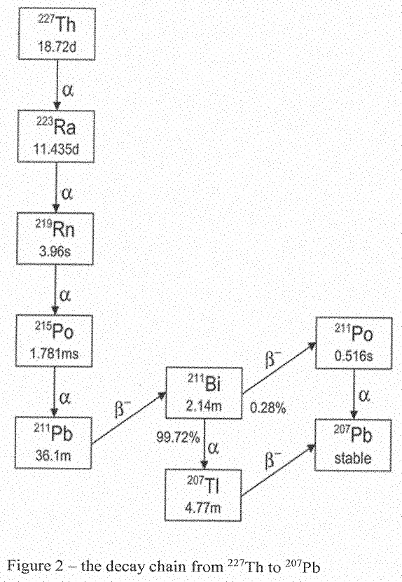

FIG. 2 Shows the radioactive decay chain of ²²⁷Th to stable ²⁰⁷Ph via ²²³Ra.

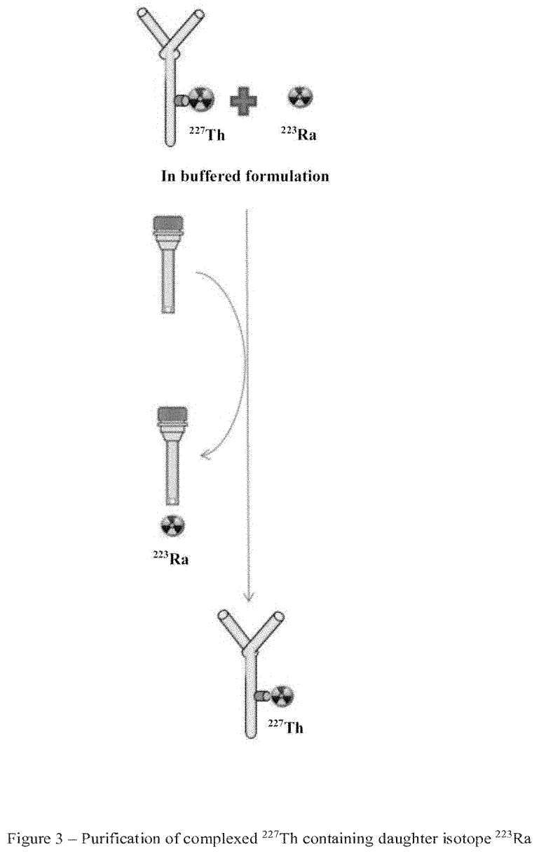

FIG. 3 Shows a schematic of the experimental steps for purification of a sample of complexed ²²⁷Th on micro-spin columns where the ²²⁷Th is partially decayed to ²²³Ra.

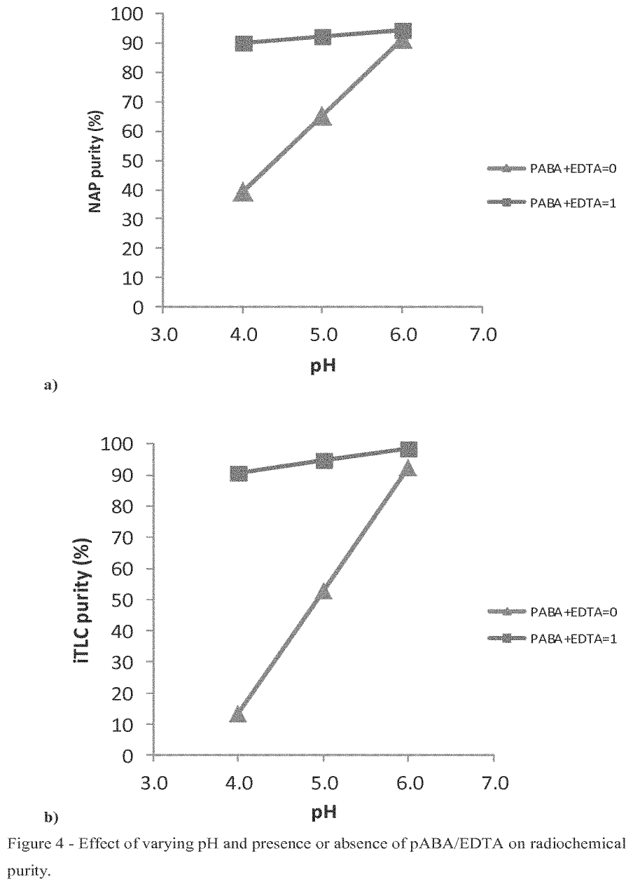

FIG. 4 Shows the effect of pH (x-axis) on radiochemical purity of a ²²⁷Th complex (y axis) in citrate buffered formulations. Plot a) contains NAP5 purity data. Plot b) is for iTLC purity data. Data series without pABA+EDTA (triangular data series) and with the presence of pABA+EDTA (square data series) are shown on each plot.

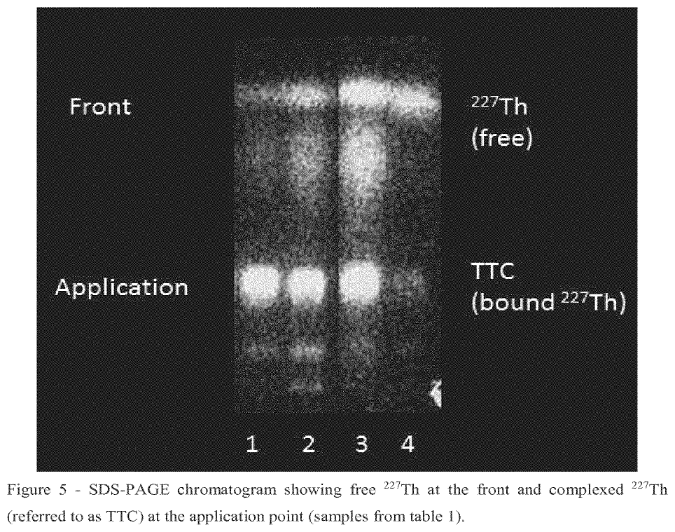

FIG. 5 SDS-PAGE chromatogram; samples 1 to 4 with application point with TTC (bound ²²⁷Th) and front line (free ²²⁷Th)

### EXAMPLES

### Materials

Sodium acetate trihydrate (≥99.0%), Sodium citrate tribasic dihydrate (≥99.0%), 4-aminobenzoic acid sodium salt (pABA, ≥99%), Edetate disodium (EDTA, meets USP testing specifications), and sodium hydroxide (98.0-100.5%) were purchased from Sigma-Aldrich. (Oslo, Norway). Metal free water (TraceSELECT) was purchased from FLUKA (Buchs, Switzerland). Sodium chloride (for analysis) and hydrochloric acid (fuming, 37%, for analysis) were purchased from Merck Millipore (Darmstadt, Germany). Citric acid monohydrate (analytical reagent) was purchased from VWR (West Chester, USA). Acetic acid (glacial, 100% anhydrous for analysis) was purchased from Merck (Darmstadt, Germany).

PSA (propylsulphonic acid) cation exchange resin based on silica was purchased from Macherey Nagel (Düren, Germany). NAP5 columns were purchased from GE Healthcare Bio-Sciences AB (Uppsala, Sweden). Pierce Micro-Spin Columns were purchased from Thermo Scientific Pierce (product number 89879 (Rockford, USA).

Trastuzumab from Herceptin® (150 mg powder for concentrate for solution for infusion) was used and is a trademark of Roche Registration Limited (Welwyn Garden City, Great Britain). To make the conjugate an in house chelator had been attached to the antibody. The resulting conjugate colloidal suspension was 5.0 mg/ml conjugate in sodium citrate buffer 0.10 M pH 5.1 and 0.90% (w/w) sodium chloride.

²²⁷Th (as thorium (IV)) in 0.05 M hydrochloric acid and metal free water (an in house product) was used as the radioactivity source. To build up to a near 1:1 ratio of ²²⁷Th and ²²³Ra (as radium (II)), ²²⁷Th was stored in order to decay for approximately one half-life of 19 days.

iTLC-SG chromatography paper impregnated with silica gel from Agilent Technologies was used for instant thin-layer chromatography (iTLC) analyses (Santa Clara, Calif.).

The following materials were used for sodium dodecyl sulphate-polyacrylamide gel electrophoresis (SDS-PAGE); LDS (4×) sample buffer and NuPage 10% tris-bis gel from Novex (Carlsbad, Calif.). MES (20×) buffer from NuPage (Carlsbad, Calif.). Instant blue from Expedeon (Cambrideshire, UK), and Precision Plus Protein dual colour Standard from BioRad (Hercules, Calif.).

### Example 1—Preparation of Buffered Formulations

Stock citrate buffers (0.10 M pH 4.0, 0.05 M pH 5.0, and 0.07 M pH 4.8) and stock acetate buffers (0.10 M pH 4.0, 0.10 M pH 6.0 and 0.10 M pH 5.0) were prepared in and diluted with metal free water (if required) to the respective buffer concentrations used in the range of the DOE. pABA (2.0 mg/ml)+EDTA. (2.0 mM) and sodium chloride were subsequently added to the respective buffered formulations containing these excipients.

The pH of the stocks and final formulations were thoroughly controlled at ambient temperature with a calibrated sevenMulti pHmeter from Mettler Toledo (Oslo, Norway).

A calibrated sevenMulti pHmeter from. Mettler Toledo (Oslo, Noway) was used to measure pH of stocks and final formulations at ambient temperature.

### Example 2 Preparation of Micro-Spin Columns with PSA Cation Exchange Resin

A 100.0 mg/ml suspension of PSA resin was prepared in metal free water. To ensure homogeneity of the suspension, a vortex mixer was used and the required volume for 15.0, 30.0, and 22.5 mg resin was added to the micro-spin columns.

For conditioning of the packed resin, 300 μl of the respective buffered formulations was added to the columns before spinning for 1 minute at 10000 ref on an Eppendorf thermomixer comfort (Hamburg, Germany (n=1, 2 or 3 for DOE samples and n=2 for center points) resulting in a dry resin bed before further use.

The columns were conditioned with 300 μl of the respective buffered formulations. The excess volume was removed by spinning for 1 minute at 10000 ref on the thermomixer resulting in dry columns (n=2 for test samples and center points).

### Example 3—Complexation and Purification

The amount of radioactivity added to each sample was approximately 250 kBq ²²⁷Th (as TTC) and 250 kBq 223Ra. Prior to use, the frozen trastuzumab-chelate conjugate colloidal suspension was allowed to equilibrate to ambient temperature. 50 μl of the conjugate was added to an Eppendorf tube with 500 kBq ²²⁷Th and 500 kBq ²²³Ra in 0.05 M hydrochloric acid (1-5 μl depending on the radioactive concentration) and mixed with 50 μl of the respective buffered formulations. The samples were then shaken 30 minutes (22° C., 750 rpm, 10 s cycles) on an Eppendorf thermomixer comfort in order to label the conjugate with decayed ²²⁷Th and form the TTC. 250 μl buffered formulation was subsequently added and mixed with the labelled conjugate (TTC) before 170 μl of this sample was added to each micro-spin column (n=1, 2 or 3 for test samples and n=2 for center points). For samples with one or three parallels, the radioactivity and volumes were adjusted as required to maintain the same conditions as for two parallels described herein. The columns were spun for 1 minute at 10000 ref on the thermomixer to elute the columns and the purified material collected in eppendorf tubes.

### Example 4—Radioassay

The amount of ²²³Ra and ²²⁷Th on the cation exchange columns and in the eluates after the separation method of Example 3 was measured before calculating the distribution of the radionuclides between the column and the eluate. HPGe spectra from a High Purity Germanium (HPGe)-detector (GEM(15) from. Qrtec (Oak Ridge, Tenn.) was used. This detector identifies and quantifies radionuclides with gamma energies ranging from approximately 30 to 1400 keV. All samples analyzed by the HPGe-detector were placed in the same position and counted for 1 min. The amount of ²²⁷Th and ²²³Ra on the columns and in the eluates after spinning was measured, and the distribution of the radionuclides between the column and the eluate was calculated by the aid of the HPGe-detector spectra. This method could be used to assay the radioisotope concentration in the eluate prior to preparing the radiopharmaceutical both to ensure a standard activity and to validate radiochemical (radioisotope) purity.

### Example 5—Stability Studies: Radiochemical Purity of the TTC

The radiochemical purity (RCP) of a radiopharmaceutical is the relationship between ²²⁷Th, in this case, present in a bound form (i.e. as TTC) to free ²²⁷Th. Since only the radionuclides ²²⁷Th and ²²³Ra are measured on the High Purity Germanium. (HPGe)-detector GEM(15), the TTC data cannot be excluded from being free ²²⁷Th. Some of the samples analysed for separation of TTC and ²²³Ra on the columns and in the eluates were therefore also analysed for RCP (gel filtration, iTLC, SDS-PAGE) with the same detector at ambient temperature after measurement of the separation of the radionuclides (within the same day).

5.1 Gel Filtration on NAP5 Columns

In order to analyse the radiochemical purity of the TTC, NAP5 columns were used (gel filtration with size exclusion). The standard procedure from the manufacturer was followed and a sample volume of 200 μl was added to the columns. HPGe-detector spectra were recorded in order to analyse the amount of TTC on the NAP5 column (n=2). See FIG. 4a)

5.2 Instant Thin-Layer Chromatography

The iTLC-SG chromatography paper was cut and dried by heating for 20-30 min in an incubator at 110-120° C. in order to be activated. A beaker was filled with approximately 0.5 cm of 0.10M citrate buffer pH 5.5 with 0.90% (w/w) sodium chloride (mobile phase). 1-8 μl of the samples (TTC purified on micro-spin columns) was applied at the origin line of the paper strip (n=2). The strip was placed vertically into the beaker, carefully avoiding any damage to the surface. When the solvent had reached the solvent front line, the strip was removed from the beaker and allowed to dry. The strip was divided into upper and lower sections by cutting the strip in half, each section of the strip was then placed in counting tubes. The activity in each half was measured separately for 5 minutes and the percentage ²²⁷Th at the front line and point of application was calculated by the aid of an Auto HPGe, Ortec gamma spectrometer with HPGe-detector (Oak Ridge, Tenn.). The results were corrected for the presence of ²²³Ra from decayed ²²⁷Th at the front line which had been building up during the time for analyses. See FIG. 4b)

5.3 Gel Electrophoresis (SDS-PAGE)

Citrate buffered samples in Table 0.1 (below) were analyzed with SDS-PAGE (n=2).

The standard procedure from the manufacturer of the NuPAGE® Bis-Tris Mini Gels was followed. MES Running Buffer was prepared by mixing 950 ml Milli Q water and 50 ml MES buffer. The samples were prepared by dilution to achieve a conjugate concentration of 1.0 mg/ml (in 0.03 M citrate buffer pH 5.5 and 0.90% w/w sodium chloride) with LDS sample buffer, Milli Q water and MES buffer. The samples were then mixed and stored on ice until, use. 5 μg of the conjugate was loaded in each well (n=2). The gel electrophoresis was run manually at a constant voltage of 200 V on the XCell SureLock Mini-Cell (Invitrogen, Carlsbad, Calif.) with Power Pac Adaptor, 4 mm and Power Pac Basic (BioRad, Hercules, Calif.). The gel was stained with Instant Blue and incubated for 60 minutes at ambient temperature. The staining reaction was stopped by washing the gel with water. The gel was then moved to a transparency film and pictures were taken. See FIG. 5

TABLE 1

| Sample | | pABA + EDTA | Spin |
|---|---|---|---|
| denomination | pH | (w/wo) | (w/wo) |
| 1 | 4.0 | w | w |
| 2 | 5.5 | wo | w |
| 3 | 4.0 | wo | wo |
| 4 | 4.0 | wo | w |

### Example 6—Separation Optimisation

A Design of Experiment (DOE) was devised to investigate and optimise the conditions for separation of ²²³Ra from ²²⁷Th on a silca/PSA micro spin column. For each buffer (citrate and acetate), the following variables were investigated:

TABLE 2

| DoE variable | Denomination | Range |
|---|---|---|
| pH citrate/acetate buffer | A | 4.0-6.0 |
| pABA + EDTA | B | w-wo |
| Buffer concentration (M) | C | 0.05-0.10 |
| Resin mass (mg) | D | 15.0-30.0 |
| Sodium chloride concentration | E | 0.45-0.90 |
| (% w/w) | | |

Each of the DoE variables was investigated using the separation and analysis methodology indicated in Examples 1 to 5. The results are shown in Table 3, which illustrate the effect of various parameters on radioisotope uptake onto PSA resin.

TABLE 3

| | Variables and correlation | | | | | | |
|---|---|---|---|---|---|---|---|
| | to uptake (positive↑ | | | Model | | | Pooled |
| | or negative↓) | | Model | 95% | Model | Pooled | 95% |
| Response | (p < 0.05) | Uptake, % | SD, % | CI | R | SD, % | CI |
| Citrate TTC | ↓A, ↑C¹, ↑D↓E¹ | 3.9-24.2 | 1.9 | ±3.8 | 0.93 | 1.3 | ±2.6 |
| Citrate ²²³Ra | ↓A, ↓C, ↑D, ↓E, AC¹ | 15.3-98.6 | 7.5 | ±15.0 | 0.96 | 2.7 | ±5.4 |
| Acetate TTC | ↓A, ↑D | 12.4-37.2 | 2.9 | ±5.8 | 0.91 | 2.2 | ±4.4 |
| Acetare ²²³Ra | ↓A, ↓B¹, ↑D | 82.3-99.3 | 3.3² | ±6.6² | 0.52 | 1.7 | ±3.4 |

¹bordeline significant,
²run with pABA/EDTA, high pH and low resin mass contribute to increased uncertainty in predicted ²²³Ra

Some examples of highly effective separation conditions were found to be:

TABLE 4

| | | pABA/EDTA | Buffer | Resin | NaCl (w/w | Predicted | Predicted |
|---|---|---|---|---|---|---|---|
| Buffer | pH | (w-w/o) | conc. (M) | mass (mg) | %) | TTC, % | ²²³Ra, % |
| Citrate | 4.0 | w | 0.05 | 15.0 | 0.45 | 13 | 82 |
| Citrate | 4.0 | w | 0.05 | 30.0 | 0.45 | 22 | 100 |
| Acetate | 4.0 | w | 0.05-0.10 | 15.0 | 0.45-0.90 | 21 | 96 |
| Acetate | 6.0 | w | 0.05-0.10 | 30.0 | 0.45-0.90 | 28 | 96 |

Where predicted TTC % is the predicted uptake of the TTC onto the resin and predicted ²²³Ra % is the predicted uptake of ²²³Ra onto the resin. High separation efficiency should combine low TTC uptake and high ²²³Ra uptake.

1: A method for the purification of complexed ²²⁷Th from a mixture comprising complexed ²²⁷Th and ²²³Ra (complexed or in solution), the method comprising:
i) preparing a first solution comprising a mixture of complexed ²²⁷Th ions and ²²³Ra ions in a first aqueous buffer;
ii) loading the first solution onto a separation material;
iii) eluting complexed ²²⁷Th from the separation material whereby to generate a second solution comprising complexed ²²⁷Th; and
iv) optionally rinsing the separation material using a first aqueous washing medium.

2: The method of claim 1, further comprising step X):
X) prior to step i), contacting ²²⁷Th ions with at least one complexing agent in solution, whereby to form at least one aqueous solution of complexed ²²⁷Th.

3: The method of claim 2, wherein the complexing agent is a chelating moiety conjugated to a targeting moiety.

4: The method of claim 1, further comprising at least one of the following steps:
v) assaying for the ²²⁷Th content of the second solution;
vi) evaporating the liquid from the second solution;
vii) forming at least one radiopharmaceutical formulation from at least a portion of the complexed ²²⁷Th contained in the second solution; and
viii) sterile filtering the radiopharmaceutical formulation.

5: The method of claim 1, wherein the first aqueous buffer is at a pH of between 3 and 6.5.

6: The method of claim 1, wherein the first aqueous buffer comprises at least one of citrate buffer, acetate buffer, and mixtures thereof.

7: The method of claim 1, wherein the first aqueous buffer solution further comprises at least one radical scavenger or at least one chelating agent, or both.

8: The method of claim 1, wherein the separation material is a silica based cation exchange resin.

9: The method of claim 8, wherein the cation exchange resin comprises at least one sulfonic acid moiety.

10: The method of claim 1, wherein the elution is under gravitational force, under centrifugal force, under gas pressure from above, under vacuum from below, or both under gas pressure from above and under vacuum from below.

11: The method of claim 10, wherein elution is under centrifugal force at a “relative centrifugal force” (RCF) of at least 5000 times the force of gravity.

12: The method of claim 10, wherein elution is under centrifugal force for a period of 10 seconds to 10 minutes.

13: The method of claim 1, wherein the method does not comprise any additional washing steps.

14: The method of claim 1, comprising washing the separation material with an aqueous washing medium.

15: The method of claim 1, additionally comprising assaying for the ²²⁷Th content of the second solution by gamma detection or gamma spectroscopy.

16: The method of claim 1 additionally comprising forming at least one radiopharmaceutical from at least a portion of the ²²⁷Th contained in the second solution comprising complexed ²²⁷Th.

17: The method of claim 16, wherein the portion of the ²²⁷Th contained in the second solution comprising ²²⁷Th is between 0.1 MBq and 20 MBq ²²⁷Th.

18: The method of any of claim 2, wherein the complexed ²²⁷Th is formed from the ²²⁷Th ions and at least one octadentate complexing agent.

19: The method of claim 18, wherein the at least one octadentate complexing agent is conjugated to a targeting moiety selected from an antibody, an antibody construct, an antibody fragment, or a construct of antibody fragments.

20: The method of claim 18, wherein the octadentate complexing agent is conjugated to a targeting moiety that has specificity for at least one target selected from “cluster of differentiation” (CD) cell surface markers.

21: The method of claim 2, wherein the contacting of step X) comprises incubating the ²²⁷Th ions with a targeting conjugate comprising a complexing agent linked to a targeting moiety, wherein such incubation is carried out at a temperature below 50° C.

22: The method of claim 21, wherein the incubation is carried out for a period of less than 2 hours.

23: The method of claim 22, wherein the incubation is carried out in the first aqueous buffer.

24: A solution of complexed ²²⁷Th comprising less than 50 KBq ²²³Ra per 1 MBq ²²⁷Th.

25: A solution of complexed ²²⁷Th comprising less than 50 KBq ²²³Ra per 1 MBq ^{227Th}, obtained by the method of claim 1.

26: A pharmaceutical composition comprising the solution of complexed ²²⁷Th of claim 24 and at least one pharmaceutically acceptable diluent.

27: A kit comprising a mixture of ²²⁷Th and ²²³Ra, a complexing agent, a first aqueous buffer solution, and a strong cation exchange resin.

28: The kit of claim 27 additionally comprising at least one of the following:
at least one sterile filter;
at least one heat resistant vessel;
at least one heating device; and
at least one pharmaceutical excipient or diluent.
